(19) United States
(12) Patent Application Publication
(10) Pub. No.: US 2004/0049192 A1
(43) Pub. Date: Mar. 11, 2004

(54) Osteosynthetic apparatus

(76) Inventor: Hirotaka Shimizu, Okayama-ken (JP)

Correspondence Address: SUGHRUE MION, PLLC, 2100 PENNSYLVANIA AVENUE, N.W., WASHINGTON, DC, 20037, US

(21) Appl. No.: 10/649,655

(22) Filed: Aug. 28, 2003

(30) Foreign Application Priority Data
Sep. 10, 2002 (JP) 2002-264734
Jan. 27, 2003 (JP) 2003-016834

(51) Int. Cl. A61B 17/58

(52) U.S. Cl. 606/62

(57) ABSTRACT

An osteosynthetic apparatus uses an intramedullary nail. The intramedullary nail is formed in a hollow shape and is provided with an outflow portion for outflow of a bone filling agent near the tip thereof. In the osteosynthetic apparatus, the intramedullary nail is provided with an insertion opening for insertion of a fixing nail in a base region thereof, the fixing nail is formed in a hollow shape and has an outflow portion for outflow of the bone filling agent near the tip thereof, and the fixing nail is inserted in the insertion opening and is fixed to the intramedullary nail. A fixing nail is of a curved shape and is rotatable in a state in which it is inserted in the fixing-nail insertion opening in the base region of the intramedullary nail. The osteosynthetic apparatus provides an enhanced fixing force of the intramedullary nail to a bone, particularly, to a bone being fragile because of osteoporosis or the like. The osteosynthetic apparatus does not have to use screws as fastening devices between the intramedullary nail and the bone, so as to reduce the invasion into the skin and soft tissue.

### BACKGROUND OF THE INVENTION

1. Field of the Invention

The present invention relates to osteosynthetic apparatus used in treatment for fractures and, more particularly, in treatment for fractures in bones suffering from osteoporosis.

2. Related Background Art

The common osteosynthesis methods conventionally applied to fractures include the following methods:

osteosynthesis with steel wires;

osteosynthesis with screws;

osteosynthesis with intramedullary nails;

osteosynthesis with screws and plates.

In these methods, screws or steel wires are thrust into the cortical layer or the cancellous tissue to fix a fracture region.

FIGS. 12A-12C show typical examples of the conventional osteosynthesis methods.

FIG. 12A shows a method of thrusting steel wires 31 from a distal region of fracture portion 30 into a bone to maintain reduction of fracture portion 30 (e.g., reference is made to Patent Document 1: Japanese Patent Application Laid-Open No. 10-33553).

FIG. 12B shows a method of maintaining reduction of fracture portion 30 with plate 32 and screws 33 (e.g., reference is made to Patent Document 2: Japanese Patent Application Laid-Open No. 2001-161704).

FIG. 12C shows a method of thrusting an intramedullary nail 34 from a proximal region of a humerus and fixing the intramedullary nail 34 at the proximal end and at the distal end with screws 33, so as to maintain the reduction of fracture portion 30 (e.g., reference is made to Patent Document 3: Japanese Patent Application Laid-Open No. 11-137566).

The conventional osteosynthesis methods described above, however, had the problem that the fixing force was weak in the case of bones suffering from osteoporosis, as frequently seen in elderly patients, and the problem that a clearance was made between the bone and the screws with a lapse of time to weaken the fixing force.

The methods using the intramedullary nail also had the problem that the invasion into the body became significant, for example, by making a plurality of incisions in the skin to permit insertion of screws.

### SUMMARY OF THE INVENTION

The present invention has been accomplished to solve the above problems in the prior art and an object of the invention is thus to provide osteosynthetic apparatus capable of achieving an enhanced fixing force of an intramedullary nail to a bone, particularly, in bones that are fragile because of osteoporosis or the like.

Another object of the present invention is to reduce the invasion into the skin and soft tissue, without use of screws as fastening devices between the intramedullary nail and the bone.

In order to achieve the above objects, an osteosynthetic apparatus according to the present invention is an osteosynthetic apparatus using an intramedullary nail, wherein the intramedullary nail is formed in a hollow shape, and comprises an outflow portion for outflow of a bone filling agent near the tip thereof, and an insertion opening for insertion of a fixing nail in a base region thereof, wherein the fixing nail is formed in a hollow shape and comprises an outflow portion for outflow of the bone filling agent near the tip thereof, and wherein the fixing nail is inserted in the insertion opening, and is fixed to the intramedullary nail by fixing means.

In the above-stated osteosynthetic apparatus of the present invention, preferably, the outflow portion of the intramedullary nail and the fixing nail near the tip thereof is comprised of a distal hole and a plurality of slits.

Another osteosynthetic apparatus according to the present invention is an osteosynthetic apparatus using an intramedullary nail, wherein the intramedullary nail is formed in a hollow shape, and comprises an outflow portion for outflow of a bone filling agent near the tip thereof, and an insertion opening for insertion of a fixing nail in a base region thereof, wherein the fixing nail is formed in a curved shape in a longitudinal direction and is rotatable in the fixing-nail insertion opening in the base region of the intramedullary nail before fixed, and wherein the fixing nail is inserted in the insertion opening, and is fixed to the intramedullary nail by fixing means.

Still another osteosynthetic apparatus according to the present invention is an osteosynthetic apparatus using an intramedullary nail, wherein the intramedullary nail is formed in a hollow shape, and comprises an outflow portion for outflow of a bone filling agent near the tip thereof, and an insertion opening for insertion of a fixing nail in the base region thereof, wherein the fixing nail is formed in a curved shape in a longitudinal direction and in a hollow shape, comprises an outflow portion for outflow of the bone filling agent near the tip thereof, and is rotatable in the insertion opening before fixed, and wherein the fixing nail is inserted in the insertion opening, and is fixed to the intramedullary nail by fixing means.

In the above-stated osteosynthetic apparatus of the present invention, preferably, the outflow portion of the intramedullary nail and the fixing nail near the tip thereof is comprised of a distal hole and a plurality of slits.

In the above osteosynthetic apparatus of the present invention, preferably, the intramedullary nail comprises an outflow portion for outflow of a fracture treatment promoter in an almost intermediate region thereof where a fracture line is located.

The above osteosynthetic apparatus according to the present invention preferably further comprises a pushing device for making a clearance between the tip of the intramedullary nail or the fixing nail and a bone.

The above solutions act as follows.

Namely, the hollow intramedullary nail is inserted into a bone and the liquid bone filling agent to harden after filling is injected from the base region into the interior of the intramedullary nail. The bone filling agent thus injected flows out of the outflow portion of the intramedullary nail near the tip to harden, so as to enhance the fixing force between the cancellous tissue and the intramedullary nail.

The fracture treatment promoter containing a substance effective in promotion of osteogenesis or tissue repair, such as growth factors of BMP, FGF, TGF-β, etc. with the effect of osteogenesis facilitation, estrogen, zinc, or the like is made to flow out of the outflow portion formed in the almost intermediate region of the intramedullary nail, to infiltrate a fracture portion and thereby facilitate the treatment of the fracture.

Furthermore, a fixing nail, or two or more fixing nails are inserted each through an insertion opening in the base region of the intramedullary nail and are fixed to the intramedullary nail. When a fixing nail of a curved shape is used, the location of the distal end of the fixing nail changes with rotation of the fixing nail. Therefore, the location of the fixing nail can be adjusted so as to match the size and shape of the portion into which the fixing nail is thrust, e.g., the head of humerus of a patient. Particularly, for example, where two fixing nails of the curved shape are used, the fixing nails can be properly rotated to change the width between the distal ends of the fixing nails.

When the fixing nail is one of a hollow shape having the outflow portion for outflow of the bone filling agent near the tip thereof, the bone filling agent of liquid to harden after filling is injected from the base region into the interior of the fixing nail. The bone filling agent flows out of the vicinity of the tip of the fixing nail to harden, thereby enhancing the fixing force between the cancellous tissue and the fixing nail.

For making a clearance for outflow of the bone filling agent, the pushing device is inserted into the hollow space in the intramedullary nail or the fixing nail to thrust its distal needle into the cancellous tissue and thereafter the pushing device is pulled out. This results in forming a clearance between the tip of the intramedullary nail or the fixing nail and a bone. The bone filling agent flows out into the clearance, so as to achieve stronger fixation.

### BRIEF DESCRIPTION OF THE DRAWINGS

FIG. 1 is a sectional view of the osteosynthetic apparatus in Embodiment 1 of the present invention as a view from slightly above the front thereof.

FIG. 2 is a top plan view of the apparatus shown in FIG. 1.

FIG. 3 is a front view showing the procedure of injecting the bone filling agent into the intramedullary nail.

FIG. 4 is a front view showing a case where the intramedullary nail is applied to a fracture in the proximal portion of a humerus.

FIG. 5 is a front view showing a case where the osteosynthetic apparatus shown in FIG. 1 is applied to a fracture in the proximal portion of a humerus.

FIG. 6 is a front view showing a case where the osteosynthetic apparatus shown in FIG. 1 is applied to a fracture in the distal portion of a radius.

FIGS. 7A and 7B are front views showing an internal cylinder and a pushing device for making a clearance between the tip of the intramedullary nail or the fixing nail and a bone, in the vicinity of holes at the tip of the intramedullary nail or the fixing nail.

FIG. 8 is a front view showing a state in which needle portions of the pushing device are projecting out of holes of the intramedullary nail.

FIG. 9 is a front view showing a state in which the bone filling agent has been injected after the formation of clearances between the osteosynthetic apparatus shown in FIG. 1 and the bone with the use of the pushing device.

FIG. 10 is a sectional view of the osteosynthetic apparatus in Embodiment 2 of the present invention as a view from slightly above the front thereof.

FIG. 11 is a top plan view of the apparatus shown in FIG. 10.

FIGS. 12A, 12B, and 12C are illustrations showing typical examples of the conventional osteosynthesis methods.

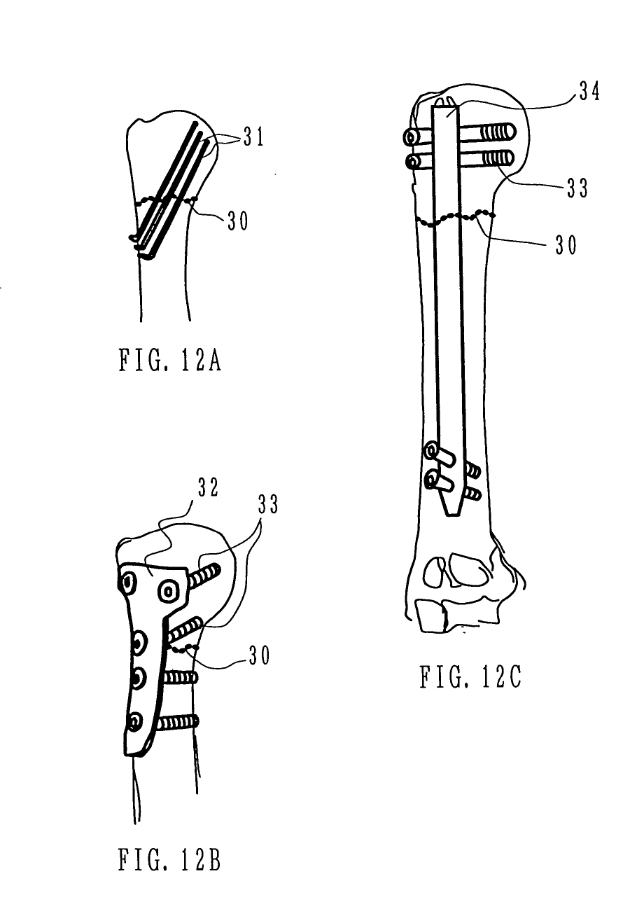

### DESCRIPTION OF THE PREFERRED EMBODIMENTS

The embodiments of the present invention will be described below on the basis of the drawings.

[Embodiment 1]

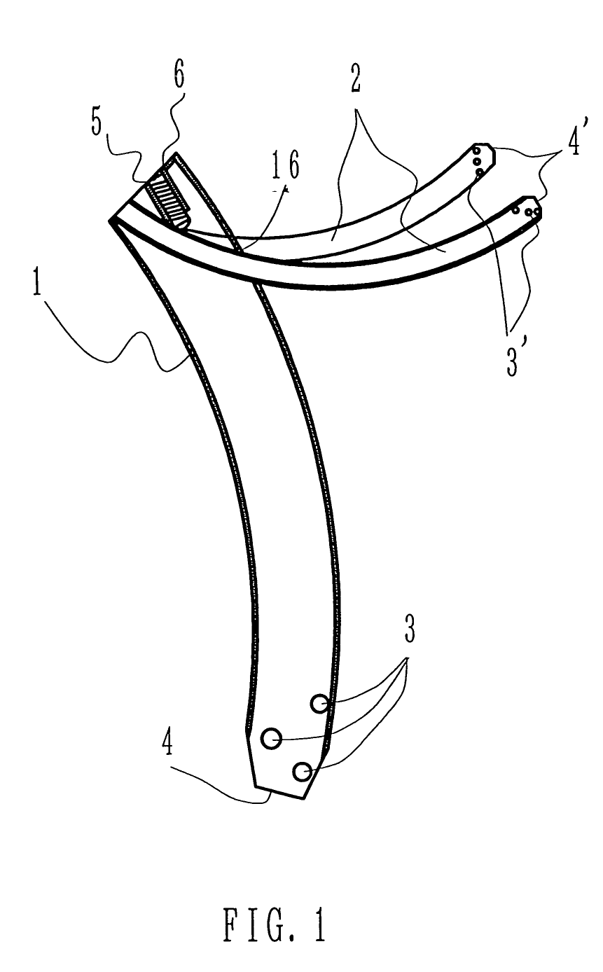

FIG. 1 to FIG. 9 show Embodiment 1 of the present invention.

FIG. 1 is a sectional view of the osteosynthetic apparatus as a view from slightly above the front thereof.

The intramedullary nail 1, which is a major component of the osteosynthetic apparatus, has a nearly circular or rectangular cross section, a hollow shape, and a predetermined length, and a number of side holes 3, in addition to a distal hole 4, are bored near the tip being the distal portion thereof. For example, in the case where the diameter of the hollow space in the intramedullary nail 1 is about 5-12 mm, the side holes 3 are approximately six to twelve holes having the diameter of approximately 1-3 mm and arranged at equal intervals in the radial direction and in the circumferential direction across a length of 3-6 cm in the distal region of the intramedullary nail 1. As detailed later, the bone filling agent is injected into the intramedullary nail 1 to flow out of the side holes 3 and distal hole 4.

In the present specification, the side holes 3 and distal hole 4 are generally called “holes” as occasion may demand. The side holes 3 may be formed in a slit shape near the tip of the intramedullary nail 1.

In FIG. 1, the intramedullary nail 1 is of an arc shape in the longitudinal direction, but it may also be of a linear shape according to the fracture portion.

Numeral 2 denotes fixing nails having a nearly circular or rectangular cross section, a hollow shape, and a curved shape or a linear shape with a pointed end in the longitudinal direction, and a number of holes 3′, in addition to a distal hole 4′, are bored near the tip being the distal end thereof, so as to permit the bone filling agent to flow out of these holes 3′, 4′, just as in the case of the intramedullary nail 1. In FIG. 1, two fixing nails 2 are used, but the number of fixing nails used may be one, or three or more according to need. Furthermore, they are of the curved shape of arc in the longitudinal direction, but they may also be of any other curved shape.

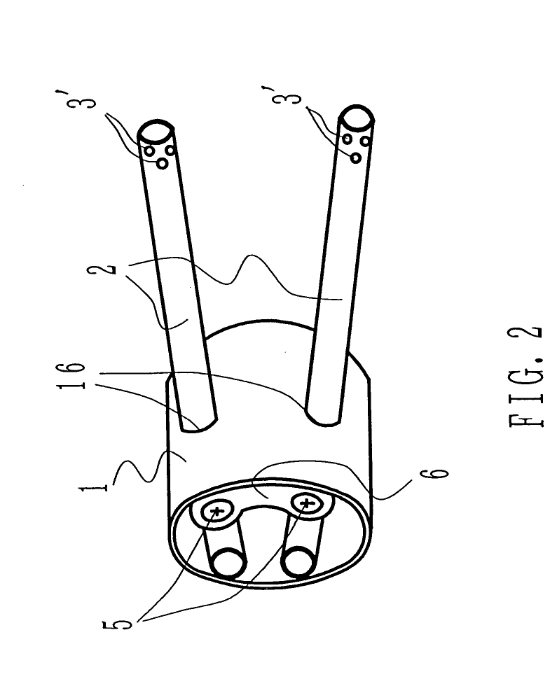

FIG. 2 is a top plan view of the apparatus shown in FIG. 1, in which two fixing nails 2 are inserted from the base region into the interior of the intramedullary nail 1 and in which the fixing nails 2 are fixed to the intramedullary nail 1 with fixing screws 5, 5 in a state where they are projecting out of the intramedullary nail 1 through nail insertion openings 16, 16 near the base region being the proximal portion of the intramedullary nail 1. Each fixing nail 2 is rotatable in an inserted state in the nail insertion opening 16. When the fixing nail 2 is one of a curved shape, the location of the distal end thereof varies with rotation of the fixing nail, so that the location of the fixing nail can be adjusted so as to match the size and shape of the part where the fixing nail is thrust, e.g., the head of a patient's humerus. The fixing screws 5, 5, as shown in FIGS. 1 and 2, are screwed into screw holes of fixing-screw attachment 6 fitted and fixed in the intramedullary nail 1, to push the fixing nails 2, 2 from above, thereby fixing the fixing nails 2, 2 to the intramedullary nail 1. The fixing-screw attachment 6 is formed separately from or integrally with the intramedullary nail 1.

The fixing nail 2 is used for stably fixing the intramedullary nail 1 to a bone, and is fixed to the intramedullary nail 1 so as to intersect with the intramedullary nail 1 at a certain angle relative to the axis of the intramedullary nail 1. The intramedullary nail 1 can also be used alone without the use of the fixing nail 2, depending upon the fracture portions.

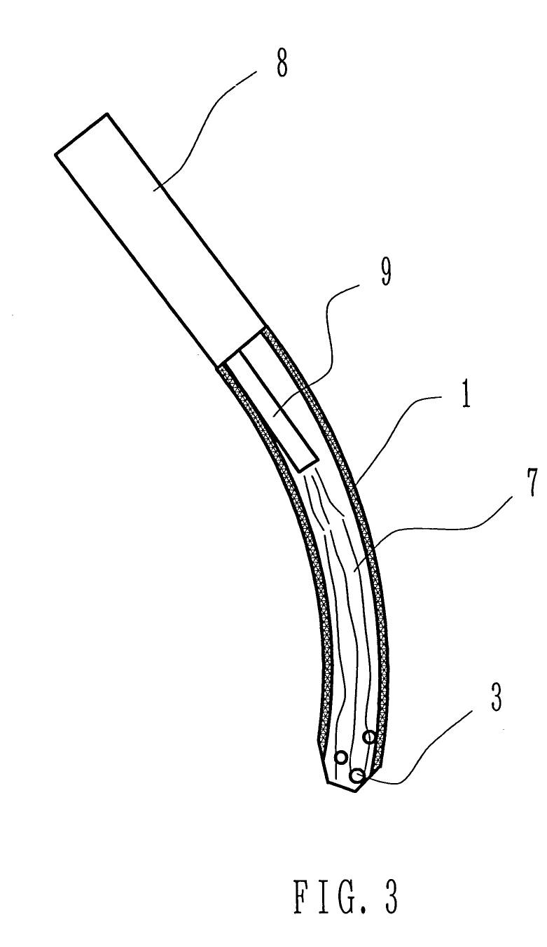

FIG. 3 shows the procedure of injecting the bone filling agent into the intramedullary nail 1. A bone filler syringe 8 is joined to the base part being the proximal portion of the intramedullary nail 1, a nozzle 9 of the syringe 8 is inserted into the hollow space of the intramedullary nail 1, and the bone filling agent 7 of liquid to harden after filling is injected thereinto. An example of the bone filling agent 7 is a substance generically known as a calcium phosphate osseous paste containing the principal components of a-tribasic calcium phosphate, tetrabasic calcium phosphate, calcium hydrogenphosphate, and hydroxyapatite (trade name: BIOPEX). The calcium phosphate osseous paste demonstrates excellent properties of biocompatibility, osseous conduction, and repair of bone, and it is initially liquid but hardens about five to ten minutes after application. Then it gradually changes the structure into hydroxyapatite in the body as time passes. The bone filling agent 7 does not have to be limited to the above example, but may be any other agent as long as it can exhibit like performance.

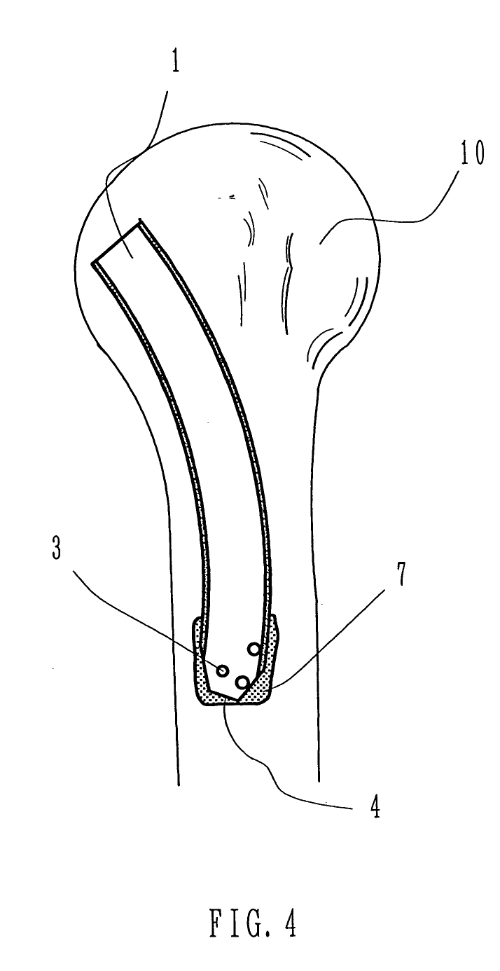

FIG. 4 shows an example where the intramedullary nail 1 is applied to a fracture in the proximal portion of humerus 10. The intramedullary nail 1 is first inserted into the humerus 10 and thereafter the bone filling agent 7 is injected into the intramedullary nail 1, whereupon the bone filling agent 7 flows out of the side holes 3, 3, . . . and distal hole 4 near the tip of the intramedullary nail 1 to harden. For this reason, the tip of the intramedullary nail 1 is fixed to the cancellous tissue by the bone filling agent 7.

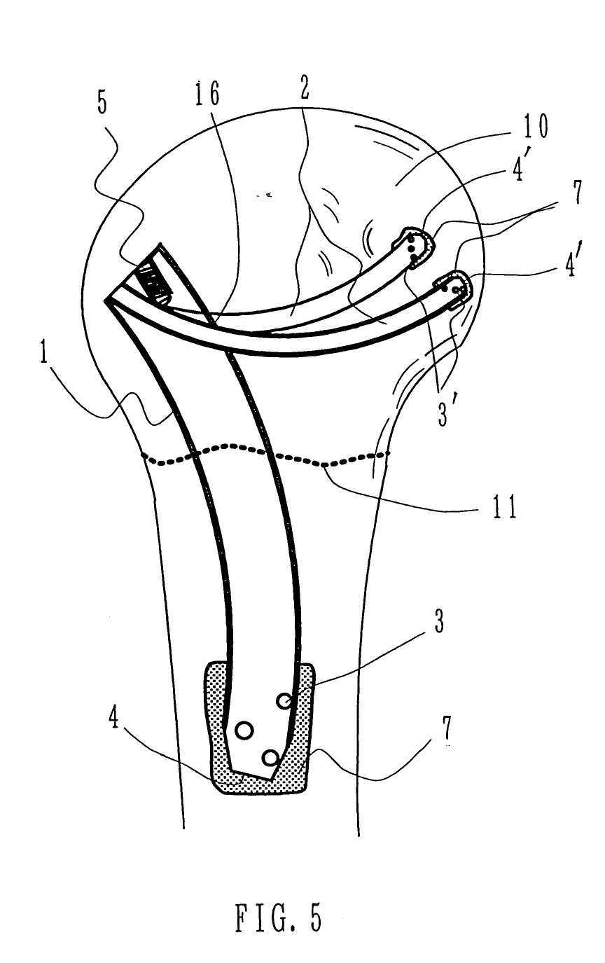

FIG. 5 shows an example where the osteosynthetic apparatus shown in FIG. 1 is applied to a fracture in the proximal portion of humerus 10. The intramedullary nail 1 is first inserted across a fracture line 11 of humerus 10 and thereafter the liquid bone filling agent 7 is injected from the base region into the interior of the intramedullary nail 1. The bone filling agent 7 thus injected flows out of the side holes 3 and distal hole 4 near the tip being the distal portion of the intramedullary nail 1 and it hardens with a lapse of time to fix the tip of the intramedullary nail 1 to the cancellous tissue. Then two fixing nails 2, 2 are inserted through the nail insertion openings 16, 16 of the intramedullary nail 1 and are fixed to the intramedullary nail 1 with fixing screws 5, 5 for fixation of the fixing nails 2. Then the liquid bone filling agent 7 is injected from the base region into the interior of the fixing nails 2, 2. The bone filling agent 7 thus injected flows out of the side holes 3′ and distal hole 4′ near the tip being the distal ends of the fixing nails 2, 2 and hardens with a lapse of time to fix the distal ends of the fixing nails 2 to the cancellous tissue. For this reason, the osteosynthetic apparatus is fixed on the both sides of the fracture line 11, whereby it can stably maintain the reduction of the fracture part. The fixation between the intramedullary nail 1 and the fixing nail 2 may also be substantialized, for example, by making use of hardening of the bone filling agent, without using the fixing screw 5.

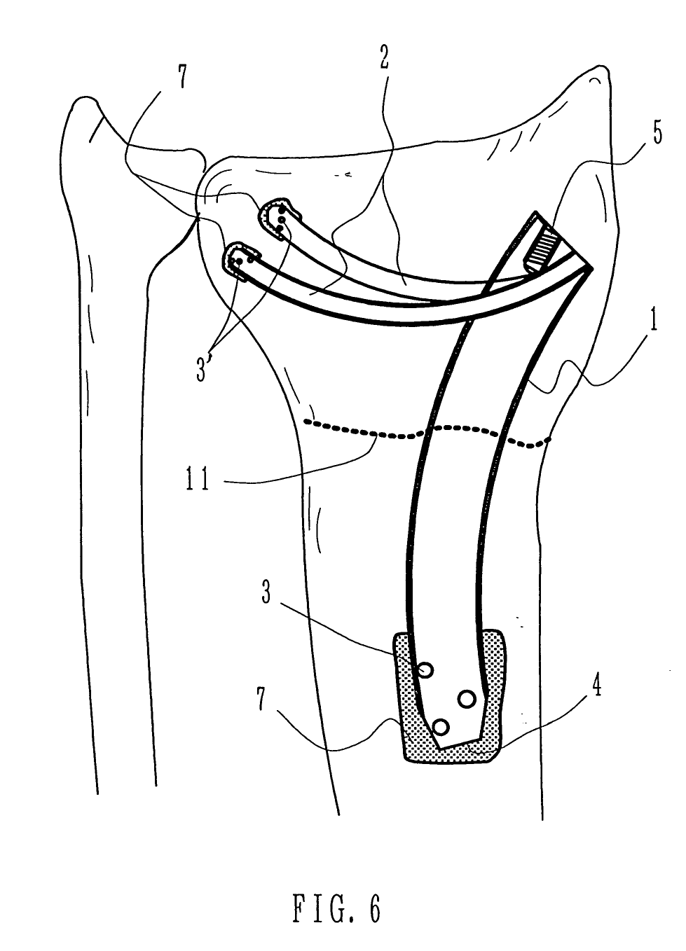

FIG. 6 shows an example where the osteosynthetic apparatus shown in FIG. 1 is applied to a fracture in the distal portion of a radius. The apparatus implements the fixation on the proximal side and the distal side with respect to the fracture line 11, so as to maintain the reduction of the fracture part with certainty.

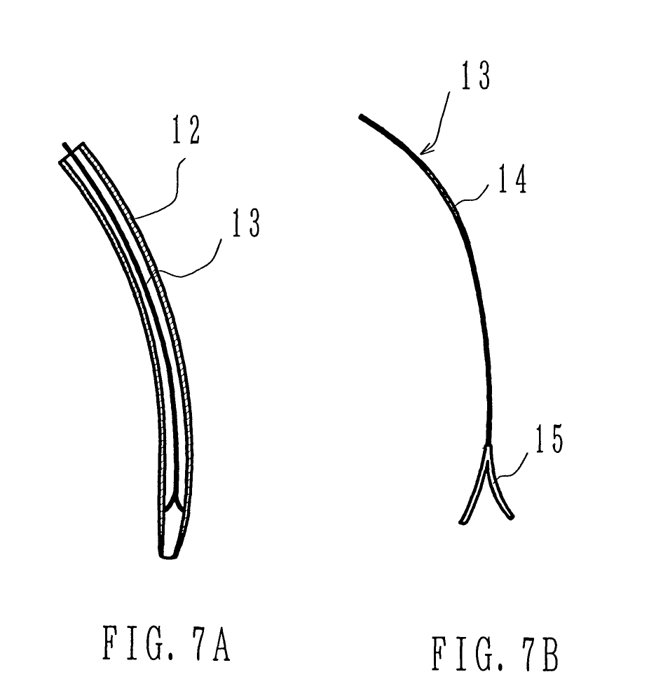

FIGS. 7A and 7B show an auxiliary device for making a clearance between the bone and the intramedullary nail 1 and between the bone and the fixing nail 2 in the vicinity of the side holes 3, 3′ near the tip of the intramedullary nail 1 and the fixing nail 2.

Numeral 12 designates an internal cylinder which can be inserted into the intramedullary nail 1. A pushing device 13 is inserted in the internal cylinder 12, as shown in FIG. 7A. As shown in FIG. 7B, the pushing device 13 is composed of flexible shaft 14, and elastically deformable, cancellous-tissue needle part 15 of a multifurcate shape, such as a bifurcate or trifurcate shape, provided at the tip of the flexible shaft 14.

The internal cylinder 12 with the pushing device 13 set therein is inserted into the intramedullary nail 1 by a predetermined length, and thereafter the pushing device 13 is pushed until the cancellous-tissue needle part 15 at the tip thereof comes to project out of the internal cylinder 12. This causes the cancellous-tissue needle part 15 to open its distal needles, and the needles project out of the intramedullary nail 1 through the holes 3, 3 thereof aligned at their respective positions, to stick in the cancellous tissue. On that occasion, the pushing device 13 can be further pushed according to need, so as to make the cancellous-tissue needle part 15 penetrate deeper into the cancellous tissue.

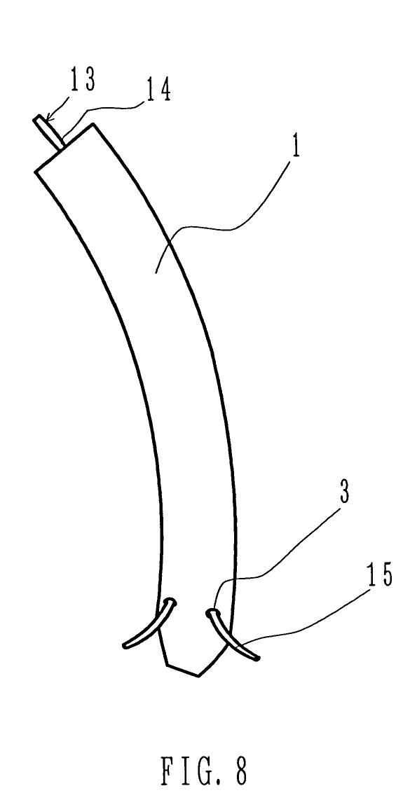

FIG. 8 shows a state in which the needle part 15 of the pushing device 13 is projecting out through the side holes 3, 3 of the intramedullary nail 1. As the cancellous-tissue needle part 15 projects out of the intramedullary nail, the cancellous-tissue needle part 15 penetrates into the cancellous tissue. Subsequently, the internal cylinder 12 with the pushing device 13 therein is removed from the intramedullary nail 1, whereupon clearances are made between the intramedullary nail 1 and the bone near the holes 3 of the intramedullary nail 1. Thereafter, the bone filling agent 7 is injected into the intramedullary nail 1, and the bone filling agent 7 flows out through the side holes 3 of the intramedullary nail 1 into the clearances. For this reason, the outflow amount and outflow area of the bone filling agent 7 increases around the intramedullary nail 1, so as to achieve a stronger fixing force.

As for the fixing nail 2, similarly as in the case of the intramedullary nail 1, clearances are made using the pushing device as an auxiliary device for making a clearance between the nail and the bone.

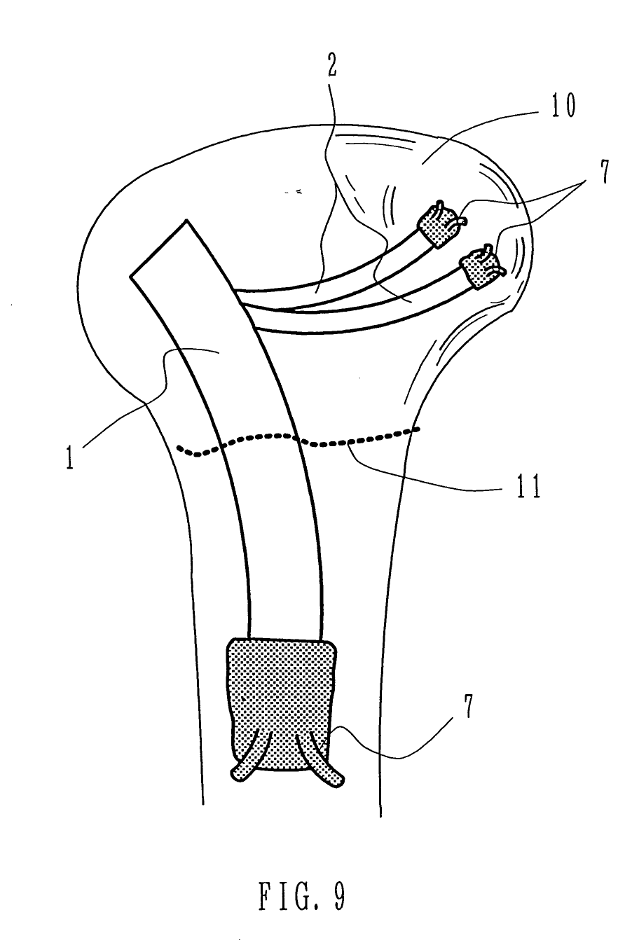

FIG. 9 shows an example in which the bone filling agent has been injected after the clearances were made between the osteosynthetic apparatus shown in FIG. 1 and the bone with the pushing device 13. It is seen that the clearances made at the tip of the intramedullary nail 1 and fixing nails 2 are filled with the bone filling agent 7 and that stronger fixation is achieved because of the increase in the outflow amount and outflow area of the bone filling agent 7.

[Embodiment 2]

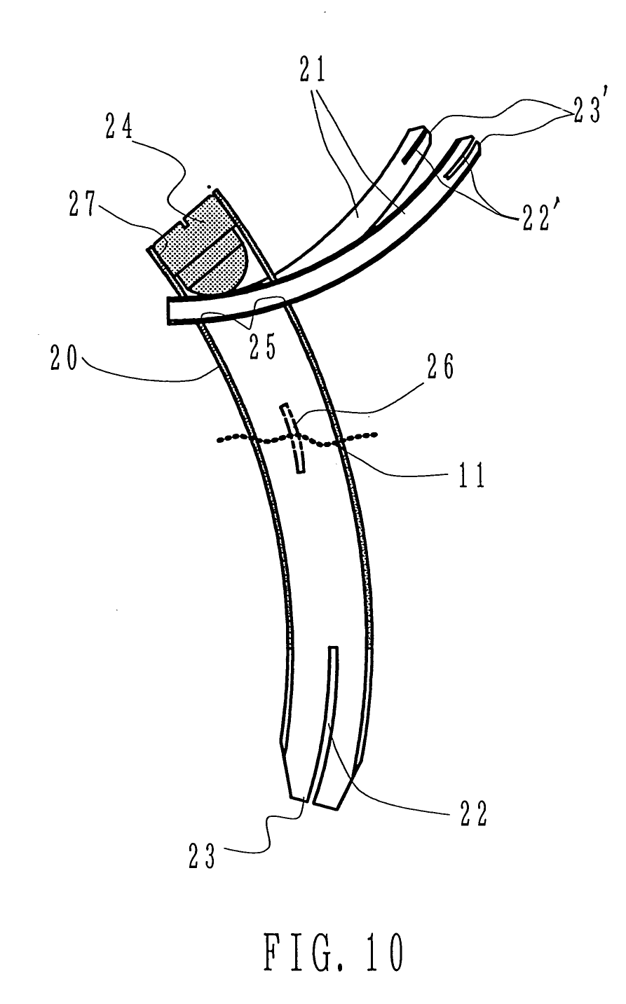

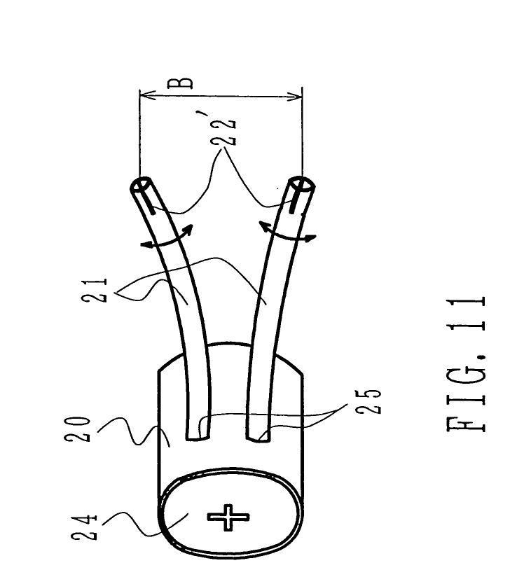

FIGS. 10 and 11 show Embodiment 2 of the present invention, wherein FIG. 10 is a sectional view of the osteosynthetic apparatus as a view from slightly above the front thereof and FIG. 11 a top plan view of the apparatus shown in FIG. 10.

The intramedullary nail 20 has a nearly circular or rectangular cross section, a hollow shape, and a predetermined length as the intramedullary nail 1 in Embodiment 1 did, and a plurality of slits 22 are formed along the axial direction, in addition to the distal hole 23, near the tip being the distal portion thereof. The slits 22 are formed in appropriate width and length according to the diameter and length of the hollow space of the intramedullary nail 20 and at equal intervals in the circumferential direction. As the bone filling agent is injected into the intramedullary nail 20, it flows out through the distal hole 23 and slits 22.

Nail insertion openings 25 are provided in a direction nearly perpendicular to the axial direction of the intramedullary nail 20 near the base region being the proximal portion of the intramedullary nail 20. Fixing nails 21, 21 have a nearly circular or rectangular cross section, a solid or hollow shape, and an arc shape with a pointed end in the longitudinal direction. The fixing nails 21, 21 are inserted into the fixing-nail insertion openings 25, and fixing screw 24 is screwed into thread part 27 formed in the base region of the intramedullary nail 20 to fix the fixing nails. FIGS. 10 and 11 show the fixing nails 21 of the hollow shape, each of which is provided with a plurality of slits 22′ along the axial direction, in addition to distal hole 23′, near the tip being the distal portion thereof so that the bone filling agent can flow out through the distal hole 23′ and slits 22′ upon injection of the bone filling agent into the fixing nail 21. It is a matter of course in the case of the fixing nail 21 of solid shape that no bone filling agent is injected into the fixing nail 21 and that the fixing nail 21 is thus provided with neither distal hole 23′ nor slits 22′.

The fixing-nail insertion openings 25 in the intramedullary nail 20 are slightly larger than the outside diameter of the fixing nail 21, so that the fixing nail 21 is inserted in a rotatable state in the fixing-nail insertion opening 25. In this configuration, since the fixing nails 21 are of the arc shape in the longitudinal direction, for example, in the case where two fixing nails 21 are used, as shown in FIG. 11, the spacing B can be adjusted between the distal ends of the fixing nails 21, 21 by rotating the fixing nails 21, 21. For example, concerning patient's humeri, the shape and size thereof enormously differ from one patient to another. In practical treatments, the locations of the fixing nails 21, 21 are adjusted by rotating the fixing nails 21, 21 so as to match the shape and size of the bone in the patient's fracture part.

The longitudinal shape of fixing nail 21 does not have to be limited to the wholly arc shape; for example, it may be a shape which is substantially linear near the base region and which increases its curvature toward the tip. The point is that the longitudinal shape of fixing nail 21 is such a curved shape as to permit adjustment of the locations of the fixing nails 21 themselves or the spacing B between the distal ends of the fixing nails 21, 21 by rotation thereof.

In addition, the intramedullary nail 20 is provided with a plurality of slits 26 for outflow of a fracture treatment promoter at equal intervals in the circumferential direction and in the axially intermediate region of the intramedullary nail 20 where the fracture line 11 is located.

The fracture treatment promoter flowing out through the slits 26 infiltrates into the fracture part to promote the treatment of the fracture.

The fracture treatment promoter can be injected by one of various methods, for example, by a method of, after injection of the bone filling agent into the intramedullary nail 20, inserting a syringe filled with the bone treatment promoter into the intramedullary nail 20 and emitting a jet of the fracture treatment promoter toward the plurality of slits 26 provided in the circumferential direction, under pressure or under action of a centrifugal force.

The combinational use of the fracture treatment promoter with the bone filling agent in this way presents the excellent effect of promoting the treatment of the fracture thanks to the infiltration of the fracture treatment promoter into the fracture part, in addition to the effect of fixing the osteosynthetic apparatus on the both sides of the fracture line 11 to stably maintain the reduction of the fracture part.

The injection of the bone filling agent into the fixing nail 21 is carried out in much the same manner as in Embodiment 1, and the auxiliary device is also used in much the same manner as in Embodiment 1, in order to make the clearances between the intramedullary nail and the bone and between the fixing nail and the bone near the slits near the tip of the intramedullary nail and the fixing nail.

For convenience' sake of description, the present invention was described in the separate forms of Embodiment 1 and Embodiment 2 as described above, but it is a matter of course that the present invention encompasses all embodied forms of carrying out the two embodiments in appropriate combination.

The present invention presents the following effects.

The bone filling agent flows out through the outflow part near the tip of the intramedullary nail to harden, whereby the fixing force of the intramedullary nail to the bone can be enhanced in fragile bones suffering from osteoporosis or the like. The fixing force of the intramedullary nail to the bone can be maintained over a long period of time after osteosynthesis.

A fixing nail, or two or more fixing nails are inserted into the base region of the intramedullary nail and fixed to the intramedullary nail, whereby the intramedullary nail is firmly fixed on the both sides of the fracture line so as to be able to stably maintain the reduction of the fracture part. In the case where the fixing nail is one of hollow shape, the bone filling agent is injected into the fixing nail whereby the fixing nail is more firmly fixed near the tip thereof with the bone filling agent.

When the fixing nail is one of curved shape, the location of the tip of the fixing nail varies with rotation of the fixing nail, so that the location of the fixing nail can be adjusted so as to match the size and shape of the part where the fixing nail is thrust, such as the head of patient's humerus. Particularly, for example, where two fixing nails are used, it is also feasible to vary the width between the distal ends of the fixing nails by properly rotating the fixing nails.

The invasion into the skin and soft tissue can be reduced, because the screws are not used as fastening devices between the intramedullary nail and the bone.

Since the intramedullary nail is provided with the outflow part for outflow of the fracture treatment promoter in the almost intermediate region thereof located at the fracture part so as to enable supply of the fracture treatment promoter into the fracture part, it is feasible to stably maintain the reduction of the fracture part and thus promote the treatment of the fracture.

What is claimed is:

1. An osteosynthetic apparatus using an intramedullary nail, wherein the intramedullary nail is formed in a hollow shape, and comprises an outflow portion for outflow of a bone filling agent near the tip thereof, and an insertion opening for insertion of a fixing nail in a base region thereof, wherein the fixing nail is formed in a hollow shape and comprises an outflow portion for outflow of the bone filling agent near the tip thereof, and wherein the fixing nail is inserted in the insertion opening, and is fixed to the intramedullary nail by fixing means.

2. The osteosynthetic apparatus according to claim 1, wherein the outflow portion of the intramedullary nail and the fixing nail near the tip thereof is comprised of a distal hole and a plurality of slits.

3. An osteosynthetic apparatus using an intramedullary nail, wherein the intramedullary nail is formed in a hollow shape, and comprises an outflow portion for outflow of a bone filling agent near the tip, and an insertion opening for insertion of a fixing nail in a base region thereof, wherein the fixing nail is formed in a curved shape in a longitudinal direction and is rotatable in the fixing-nail insertion opening in the base region of the intramedullary nail before fixed, and wherein the fixing nail is inserted in the insertion opening, and is fixed to the intramedullary nail by fixing means.

4. An osteosynthetic apparatus using an intramedullary nail, wherein the intramedullary nail is formed in a hollow shape, and comprises an outflow portion for outflow of a bone filling agent near the tip thereof, and an insertion opening for insertion of a fixing nail in the base region thereof, wherein the fixing nail is formed in a curved shape in a longitudinal direction and in a hollow shape, comprises an outflow portion for outflow of the bone filling agent near the tip thereof, and is rotatable in the insertion opening before fixed, and wherein the fixing nail is inserted in the insertion opening, and is fixed to the intramedullary nail by fixing means.

5. The osteosynthetic apparatus according to claim 4, wherein the outflow portion of the intramedullary nail and the fixing nail near the tip thereof is comprised of a distal hole and a plurality of slits.

6. The osteosynthetic apparatus according to claim 1 or claim 3, wherein the intramedullary nail comprises an outflow portion for outflow of a fracture treatment promoter in an almost intermediate region thereof where a fracture line is located.

7. The osteosynthetic apparatus according to claim 1 or claim 3, comprising a pushing device for making a clearance between the tip of the intramedullary nail or the fixing nail and a bone.
